(19) United States
(12) Patent Application Publication
(10) Pub. No.: US 2024/0309035 A1
(43) Pub. Date: Sep. 19, 2024

(54) ENZYMATIC SYNTHESIS OF HOMOGENEOUS CHONDROITIN SULFATE OLIGOSACCHARIDES

(71) Applicant: The University of North Carolina at Chapel Hill, Chapel Hill, NC (US)

(72) Inventors: Jian Liu, Chapel Hill, NC (US); Jian Liu, Chapel Hill, NC (US); Guowei Su, Chapel Hill, NC (US)

(21) Appl. No.: 18/674,521

(22) Filed: May 24, 2024

Related U.S. Application Data
(62) Division of application No. 16/625,342, filed on Dec. 20, 2019, now Pat. No. 11,993,627.
(60) Provisional application No. 62/528,243, filed on Jul. 3, 2017.

(51) Int. Cl. C07H 3/06; C07H 11/00; C08B 37/00; C12N 9/10; C12P 19/26

(52) U.S. Cl. CPC C07H 3/06; C07H 11/00; C08B 37/0063; C08B 37/0069; C12N 9/1048; C12N 9/13; C12P 19/26

(57) ABSTRACT

Methods of synthesizing chondroitin sulfate oligosaccharides are provided. Enzymatic schematic approaches to synthesizing structurally defined homogenous chondroitin sulfate oligosaccharides at high yields are provided. Synthetic chondroitin sulfate oligosaccharides ranging from 3-mers to 15-mers are provided.

### CROSS-REFERENCE TO RELATED APPLICATIONS

This application is a divisional application of U.S. patent application Ser. No. 16/625,342, filed Dec. 20, 2019, which is a national phase patent application under 35 U.S.C. Section 371 of PCT International Patent Application No. PCT/US2018/040774, filed Jul. 3, 2018, which claims priority to and benefit of U.S. Provisional Patent Application Ser. No. 62/528,243, filed Jul. 3, 2017. The disclosure of each of these applications is incorporated herein by reference in its entirety.

### GRANT STATEMENT

This invention was made with government support under Grant Numbers GM102137 and HL094463 awarded by the National Institutes of Health. The government has certain rights in the invention.

### TECHNICAL FIELD

The subject matter disclosed herein relates generally to synthesis of chondroitin sulfate oligosaccharides. More particularly, the subject matter disclosed herein relates to methods of enzymatic synthesis of homogenous chondroitin sulfate oligosaccharides, and homogenous chondroitin sulfate oligosaccharides products thereof.

### BACKGROUND

Chondroitin sulfates (CS) are sulfated polysaccharides and widely present on the mammalian cell surface and in the extracellular matrix. CS is known to be involved in cancer metastasis¹, parasitic infections²⁻³ and neuron growth inhibition after injury⁴⁻⁶. The sulfation patterns in CS dictate the binding affinity to the protein targets to manifest the selectivity in biological functions⁷. A 6-O-sulfated N-acetyl galactosamine (GalNAc6S) containing CS facilitates the infection of B. burgdorferi to cause lyme disease⁸, and 4-O-sulfated N-acetyl galactosamine (GalNAc4S) involves in P. falciparum infection to cause malaria². A domain containing 4,6 disulfated N-acetyl galactosamine (GalNAc4S6S) residues is proved to be necessary to direct neuronal signaling and inhibit axon growth^{6, 9}. CS in conjunction with glucosamine is prescribed as a treatment for osteoarthritis in Asia and Europe and available as a nutraceutical supplement in US; however, the efficacy remains controversial¹⁰⁻¹¹. Bikunan, a special form CS, exhibits defined saccharide sequences¹²; however, the CS isolated from natural human or animal tissues are present in a mixture of different sulfated saccharides and sizes. To isolate a polysaccharide with a single sulfated saccharide sequence and defined length is technically demanding. The lack of structurally homogeneous or monodisperse CS is the major roadblock that hinders CS research.

There remains a need for an effective method to synthesize structurally defined CS oligosaccharides to bridge the gap for investigating the structure-activity relationship (SAR). Moreover, there remains a need for the development of methods to synthesize homogeneous CS oligosaccharides at an efficiency high enough to support a wide range of biological evaluations.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are methods of synthesizing a synthetic chondroitin sulfate oligosaccharide, comprising providing a chondroitin backbone, and performing at least two of the following enzymatic reaction steps: an elongation step to add a GalNAc residue using a glycosyltransferase from E. coli K4 (KfoC), an elongation step to add a GlcA residue using KfoC, a 6-O-sulfation step using chondroitin sulfate 6-O-sulfotransferase (CS6OST) and sulfate donor 3′-phosphoadenosine 5′-phosphosulfate (PAPS), and a 4-O-sulfation step involving chondroitin sulfate 4-O-sulfotransferase (CS4OST) and PAPS, whereby a synthetic chondroitin sulfate oligosaccharide is synthesized.

In some embodiments, provided herein are synthetic chondroitin sulfate oligosaccharides produced by the disclosed methods. In some embodiments, provided herein are libraries or panels of synthetic chondroitin sulfate oligosaccharides produced by the disclosed methods. In some embodiments, provided herein are synthetic chondroitin sulfate oligosaccharides comprising a 4-O-sulfated and/or 6-O-sulfated chondroitin oligosaccharide having a size ranging from a 3-mer to a 15-mer. In some embodiments, provided herein are synthetic chondroitin sulfate oligosaccharides comprising are selected from chondroitin sulfate A (CS-A), chondroitin sulfate C (CS-C), chondroitin sulfate D (CS-D) and chondroitin sulfate E (CS-E).

Accordingly, it is an object of the presently disclosed subject matter to provide synthetic chondroitin sulfate oligosaccharides and methods for synthesizing synthetic chondroitin sulfate oligosaccharides.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 is a schematic illustration of fifteen exemplary CS compounds synthesized according to the presently disclosed methods;

FIGS. 2A and 2B are schematic illustrations of enzymatic method steps and routes for synthesizing CS compounds as disclosed herein;

FIGS. 3A and 3B depict the results of structural analysis of one exemplary CS compound (compound 9), with FIG. 3A being a schematic illustration of compound 9 with chemical shifts, and FIG. 3B being a plot of heteronucluear multiple-bond correlation spectroscopy (HMBC) of 2D-NMR;

FIG. 4 is a schematic illustration of exemplary CS compounds, namely CS-A, CS-C and CS-E, synthesized according to the presently disclosed methods, and shown in an abbreviated chemical structure format;

FIGS. 5A and 5B are schematic illustrations of the full chemical structures of exemplary CS-A compounds;

FIGS. 6A and 6B are schematic illustrations of the full chemical structures of exemplary CS-C compounds;

FIGS. 7A and 7B are schematic illustrations of the full chemical structures of exemplary CS-E compounds; and

FIG. 8 is a schematic illustration of a synthetic route to synthesize CS-E from CS-A.

### DETAILED DESCRIPTION

The presently disclosed subject matter now will be described more fully hereinafter, in which some, but not all embodiments of the presently disclosed subject matter are described. Indeed, the disclosed subject matter can be embodied in many different forms and should not be construed as limited to the embodiments set forth herein; rather, these embodiments are provided so that this disclosure will satisfy applicable legal requirements.

Chondroitin sulfate (CS) is a sulfated polysaccharide that plays essential physiological roles. Disclosed herein are novel enzyme-based methods for synthesizing CS compounds and libraries of CS, including for example a library of CS oligosaccharides comprising a plurality of different CS oligosaccharides ranging from 5-mers to 19-mers. The exemplary library of CS disclosed herein includes 4-O-sulfated and 6-O-sulfated oligosaccharides with the size ranging from trisaccharide to nonasaccharide. Also disclosed herein is the synthesis of unnatural 6-O-sulfated CS pentasaccharides containing either a 6-O-sulfo 2-azido galactosamine or a 6-O-sulfo galactosamine residue. As disclosed herein, the availability of structurally defined CS oligosaccharides, based on the methods and systems disclosed herein, offers a novel approach to investigate the biological functions of CS. In some embodiments, an unnatural CS can be any CS compound where the NHAc has been changed to another moiety, such as an azide or trifluoro, for example. Moreover, a natural CS can be any CS compound that includes a N-acetylgalactosamine (GalNAc). Thus, in some embodiments any CS compound where GalNAc has been modified, changed or removed can be considered unnatural.

To isolate a polysaccharide with a single sulfated saccharide sequence and defined length is technically demanding. The lack of structurally homogeneous or monodisperse CS is a major roadblock that hinders CS research. Thus, until the present disclosure there remained a need for an effective method to synthesize structurally defined CS oligosaccharides to bridge the gap for investigating the structure-activity relationship (SAR). Moreover, the instant disclosure fills a void for methods to synthesize homogeneous CS oligosaccharides at an efficiency high enough to support a wide range of biological evaluations.

Chondroitin sulfates are sulfated polysaccharides and widely present on the mammalian cell surface and in the extracellular matrix. The sulfation patterns in CS dictate the binding affinity to the protein targets to manifest the selectivity in biological functions. For example, a 6-O-sulfated N-acetyl galactosamine (GalNAc6S) containing CS can facilitate the infection of B. burgdorferi to cause lyme disease⁸, and 4-O-sulfated N-acetyl galactosamine (GalNAc4S) can be involved in P. falciparum infection to cause malaria².

Chondroitin sulfates contain the repeating disaccharide unit of [symbol] 1→3-linked glucuronic acid (GlcA) and N-acetylgalactosamine (GalNAc) disaccharide, →4)GlcA [symbol] (1→3) GalNAc [symbol] (1→. Both GlcA and GalNAc residues carry sulfo groups, giving rise to different types of CS. Chondroitin sulfate A (CS-A) contains 4-O-sulfated GalNAc (GalNAc4S) residues, chondroitin sulfate C (CS-C) contains 6-O-sulfated GalNAc (GalNAc6S), chondroitin sulfate D (CS-D) contains 2-O-sulfated GlcA and chondroitin sulfate E (CS-E) contains 4,6-O-disulfated GalNAc. Specialized CS sulfotransferases, including 4-O-sulfotransferase (CS4OST), 6-O-sulfotransferase (CS6OST), 2-O-sulfotransferase and GalNAc4S-6-O-sulfotransferase, participate in the biosynthesis of CS.

Unlike chemical synthesis of CS oligosaccharides, provided herein are CS biosynthetic enzymes and methods of using the same to synthesize CS oligosaccharides at a rate, cost and efficiency never before seen. Until the instant disclosure, enzymatic methods, systems and procedure have clearly been unable to prepare homogeneous CS oligosaccharides at an efficiency sufficient to support a wide range of biological evaluations.

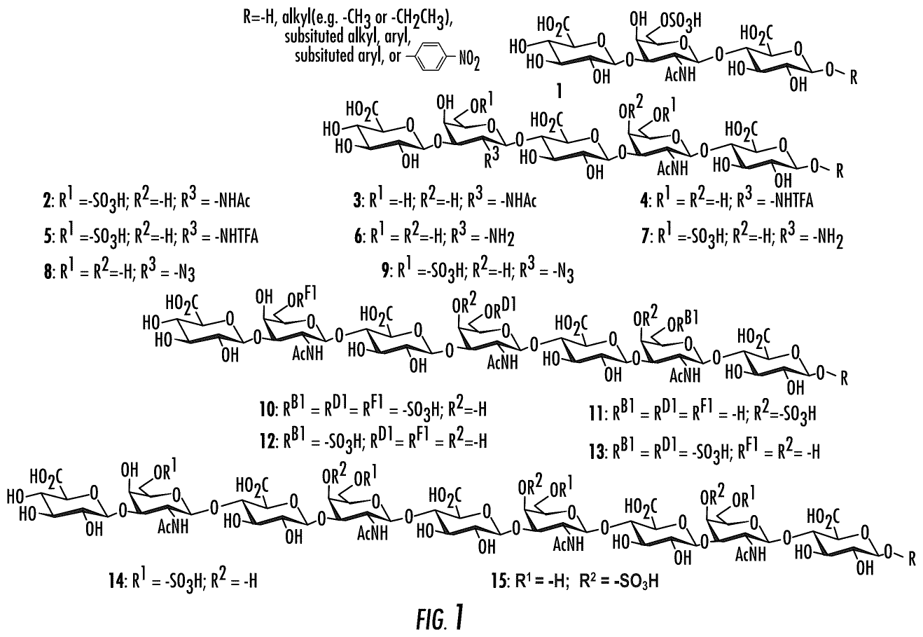

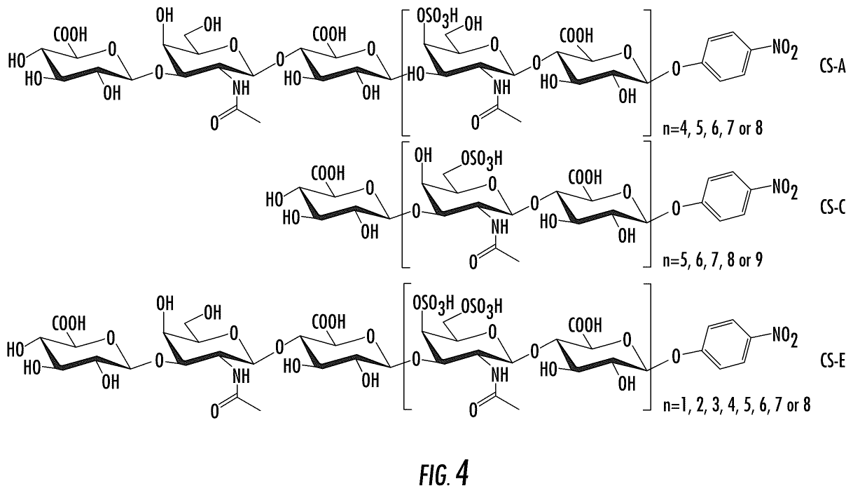

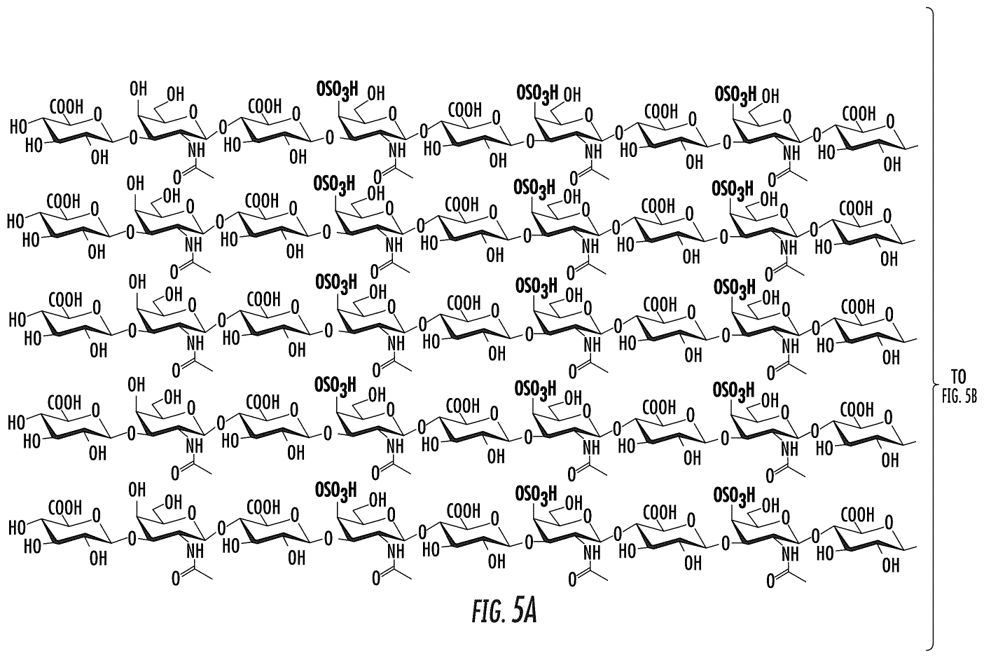

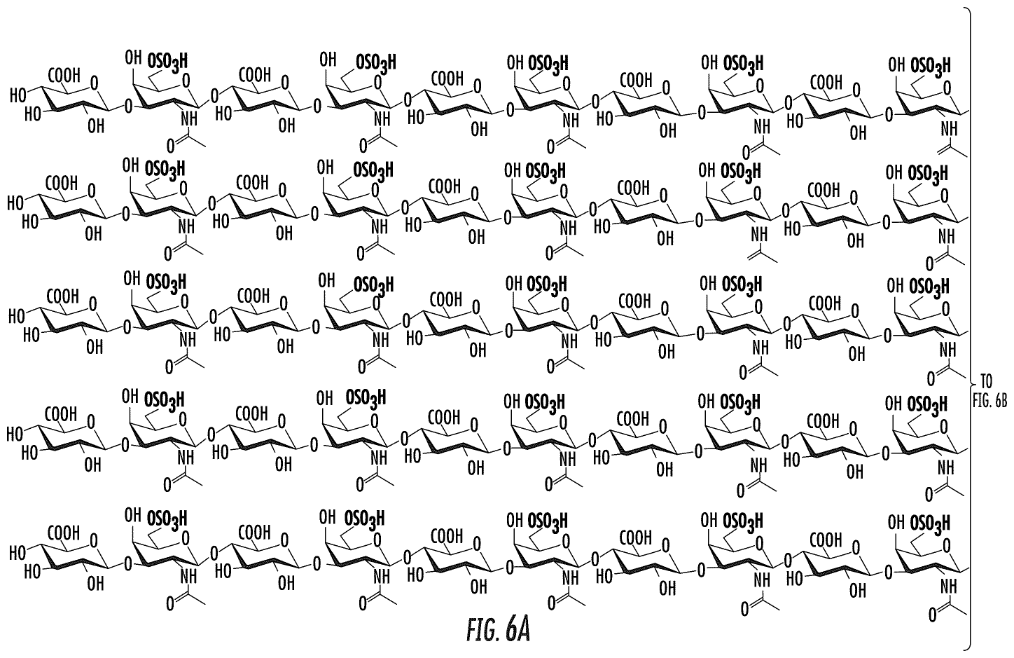

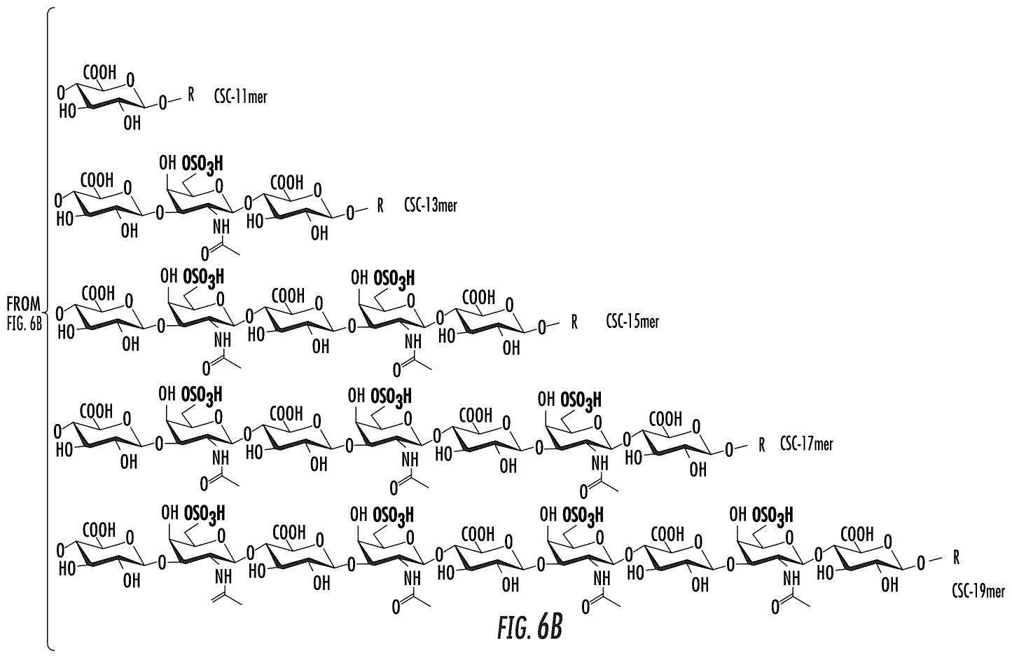

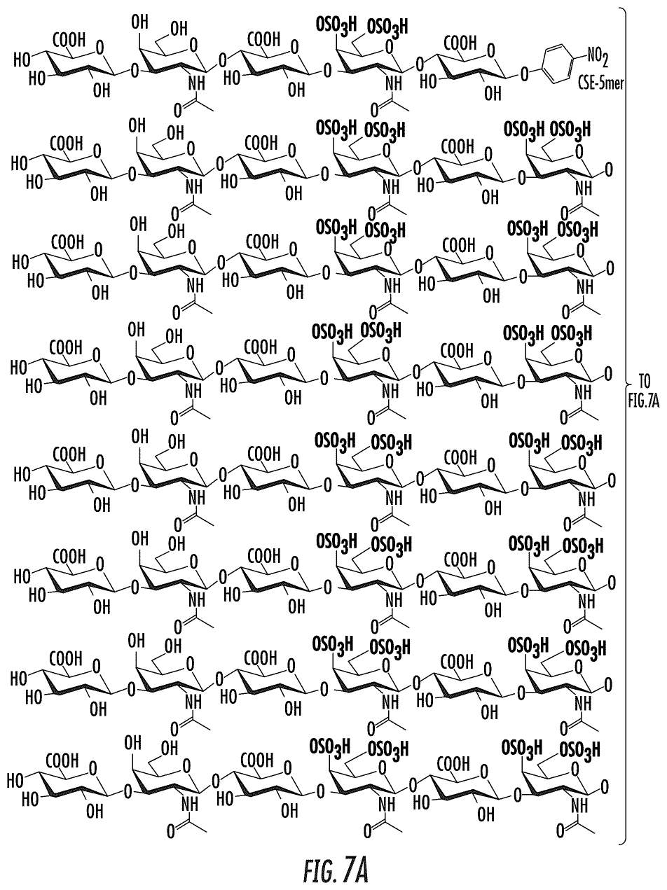

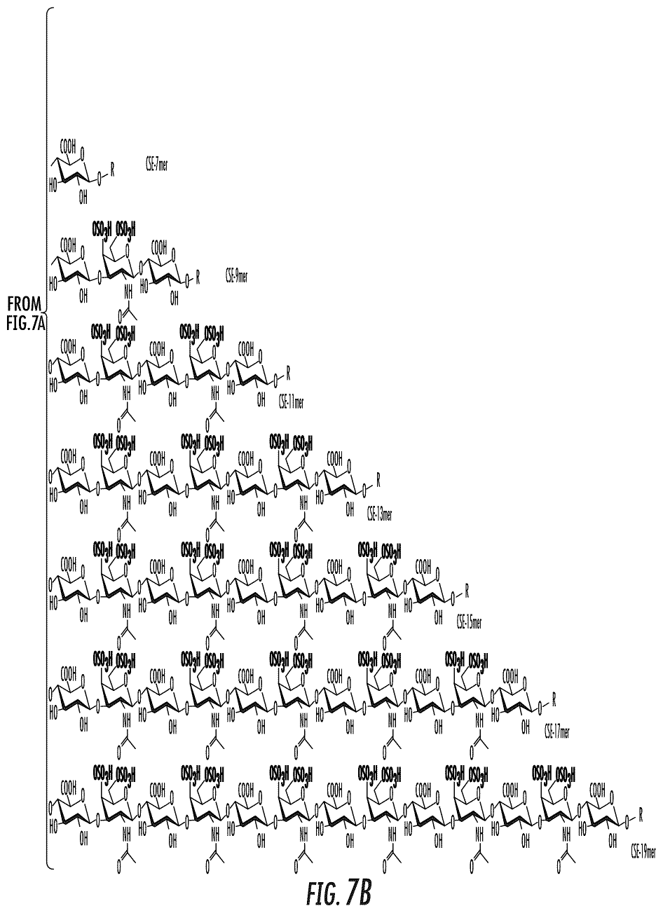

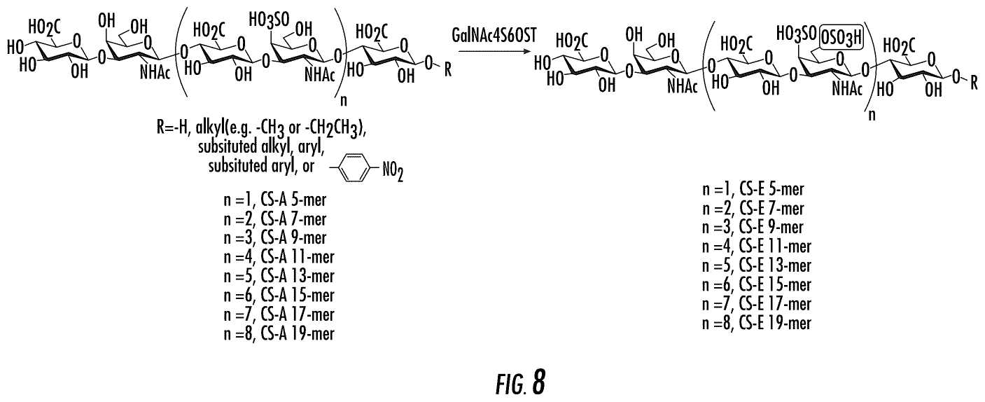

A plurality of CS oligosaccharides, including 15 CS oligosaccharides up to 9-mer, were synthesized using the disclosed methods at the scale of about 4 to about 30 mg (see, e.g., FIG. 1 and Table 1). Even longer and/or larger CS oligosaccharides, including up to 19-mer, were also synthesized using the disclosed methods, as shown and illustrated in FIGS. 4, 5A, 5B, 6A, 6B, 7A, 7B, and 8. These CS compounds are exemplary only and not intended to be limited since one of ordinary skill in the art could synthesize any number of CS compounds using the disclosed enzymatic methods, systems and schematics without departing from the scope of the instant disclosure.

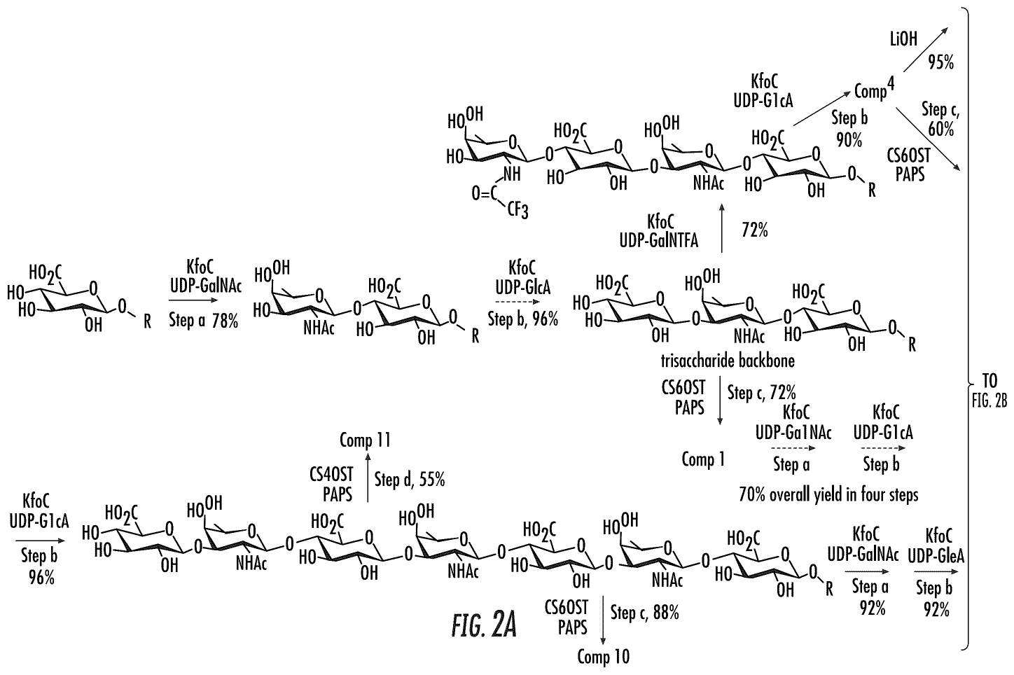

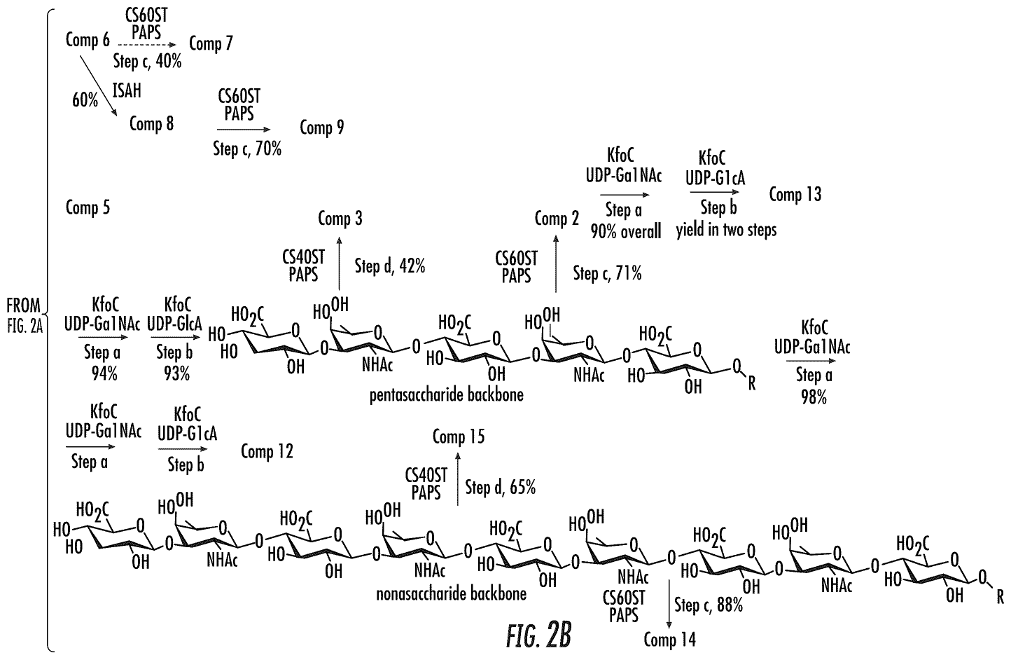

In some embodiments, the disclosed methods provide for both the synthesis of nonsulfated chondroitin backbones and sulfations of the backbones to yield the final products. Thus, a CS backbone can in some embodiments be any CS compound without one or more sulfation groups, i.e. a nonsulfated CS compound can be considered a CS backbone. The synthesis of nonsulfated chondroitin was accomplished using a bacterial glycosyltransferase from E. coli K4 strain, known as KfoC. The enzyme transfers both UDP-GalNAc and UDP-GlcA to the acceptor to form chondroitin. The recombinant KfoC was readily obtained through the expression in high yield, offering an effective way to synthesize chondroitin oligosaccharides²². However, challenges in preparing oligosaccharides in large quantity and high purity were encountered. First, KfoC displays a promiscuous donor substrate specificity towards N-acetyl hexosamine donors. It transfers a GalNAc residue from UDP-GalNAc to the acceptor, but also transfers an N-acetyl glucosamine (GlcNAc) from UDP-GIcNAc, which is not present in CS²³. This property raised serious concerns for the purity of the oligosaccharide products if a donor substrate UDP-GalNAc is contaminated with UDP-GlcNAc, causing an incorporation of a GlcNAc residue into the chondroitin backbone. Second, the cost of UDP-GalNAc is high, about $16,000/mmole from a commercial source. Such price is prohibitively expensive to conduct gram-scale synthesis. To overcome the limitation, UDP-GalNAc was synthesized from GalNAc using two enzymes including N-acetylhexosamine kinase (NAHK) and mammalian GalNAc pyrophosphorylase AGX1. This success eliminated the risk of contamination of UDP-GIcNAc as well as reduced the cost of UDP-GalNAc by about 2500-fold. Using the scheme described in FIGS. 2A and 2B, for example, gram-scale of chondroitin backbone were routinely synthesized to prepare different CS oligosaccharides.

KfoC enzyme is capable of transferring a GalNTFA residue from UDP-GalNTFA, where GalNTFA represented N-trifluoroacetyl galactosamine, into the chondroitin backbone to yield compound 4 (FIG. 2). The introduction of a GalNTFA residue into the backbone offered the flexibility to convert it to a GalNH₂ or a GalN_{AZ} residue as demonstrate in the synthesis of compound 6 and 8, respectively. Neither GalNH₂ nor GalN_{AZ} residue was reported in CS isolated from natural sources, and these structures have not been synthesized previously. The data provided herein demonstrate that the ability to synthesize structurally diversified chondroitin backbone structures by exploiting the substrate specificity of the enzyme.

4-O-sulfated oligosaccharides were successfully synthesized using CS4OST. In this study, three oligosaccharides, including 4, 11 and 15, were synthesized. Partially 4-O-sulfated products were obtained. Structural analysis of 4, 11, 15 by NMR revealed that the GalNAc residue that is near the nonreducing end was devoid of 4-O-sulfation. The inability of sulfation at the GalNAc near the nonreducing end was attributed to the substrate specificity of CS4OST.

Eight exemplary 6-O-sulfated oligosaccharides were synthesized, including for example CS compounds 1, 2, 5, 7, 9, 10, 13 and 14 (see, e.g. Table 1) using CS6OST. Depending on the positions of 6-O-sulfation, different synthetic schemes were used. Compounds 1, 2, 10 and 14 are fully 6-O-sulfated oligosaccharides ranging from tri-saccharide to nona-saccharide. The synthesis was achieved by incubating the chondroitin backbone (tri- to nona-saccahrides) with CS6OST (FIGS. 2A and 2B).

The Synthetic scheme of CS oligosaccharides using enzymes in FIGS. 2A and 2B can in some embodiments comprise the following steps: step a can comprise an elongation step to add a GalNAc residue involving KfoC, a glycosyltransferase from E. coli K4; step b can also comprise an elongation step to add a GlcA residue using KfoC; step c can comprise a 6-O-sulfation step involving chondroitin sulfate 6-O-sulfotransferase (CS6OST) and sulfate donor 3′-phosphoadenosine 5′-phosphosulfate (PAPS); and step d can comprise a 4-O-sulfation step involving chondroitin sulfate 4-O-sulfotransferase (CS4OST) and PAPS. In some embodiments KfoC can also transfer a GalNTFA residue to a trisaccharide backbone to yield a tetrasaccharide. An exemplary yield from each reaction step is indicated in FIGS. 2A and 2B. ISAH represents imidazole-1-sulfonyl azide.

The syntheses of partially 6-O-sulfated heptasaccharides, including compounds 12 and 13, were achieved using different starting 6-O-sulfated oligosaccharides. To synthesize compound 12, a 6-O-sulfated trisaccharide (compound 1) was exposed to elongation reaction using KfoC (FIGS. 2A and 2B). To prepare compound 13, the starting material was compound 2. The rearrangement of the sequence of enzyme modification to achieve partially 6-O-sulfated oligosaccharides demonstrate the flexibility of the enzymatic synthesis to prepare structurally diversified CS-C oligosaccharides.

The chondroitin backbones containing a GalNTFA, GalNH₂ or GalN_{AZ} residue were employed to prepare the 6-O-sulfated oligosaccharides, compound 5, 7 and 9 (FIGS. 2A and 2B). The success of synthesis of compound 5, 7, and 9 demonstrated that CS6OST displays the tolerance on the amino substitution of the GalNAc residue. However, the enzyme appears to be very specific towards the chondroitin backbone. Using a heparan hexasaccharide substrate, no reaction was found where the repeating disaccharide unit is [symbol] [symbol] 1,4-linked GlcA and N-acetylated glucosamine (GlcNAc),→4)GlcA [symbol] (1→4) GlcNAc [symbol] (1→.

Based on the instant disclosure it has been discovered that at least one desirable factor to synthesize CS-A and CS-C oligosaccharides was to obtain high quality recombinant CS4OST and CS6OST. The enzymes were obtained from insect cells using baculovirus expression approach. In some embodiments it was desirable to remove the viral chondroitinase contamination from enzyme preparations. The chondroitinase degraded chondroitin backbone substrate, leaving no products. The expression of CS6OST in E. coli was unsuccessful, possibly due to the fact that glycosylation is required for the sulfotransferase activity.

TABLE 1
Summary of synthetized CS oligosaccharides up to 9-mer

| | | | | Measured | Calculated |
|---|---|---|---|---|---|
| | Abbreviated saccharide | Amount | Purity¹ | Molecular | molecular |
| Compound | sequence | (mg) | (%) | Mass (Da) | Mass (Da) |
| 1 | GlcA-GalNAc6S-GlcA- | 4 | 96% | 774.0 ± 0.6 | 774.6 |
| | pNP | | | | |
| 2 | GlcA-GalNAc6S-GlcA- | 15 | >98% | 1233.9 ± 0.7 | 1234.0 |
| | GalNAc6S-GlcA-pNP | | | | |
| 3 | GlcA-GalNAc-GlcA- | 9 | >98% | 1153.8 ± 0.7 | 1153.9 |
| | GalNAc4S-GlcA-pNP | | | | |
| 4 | GlcA-GalNTFA-GlcA- | 30 | 59% | 1127.9 ± 0.7 | 1127.8 |
| | GalNAc-GlcA-pNP² | | | | |
| 5 | GlcA-GalNTFA6S-GlcA- | 4 | 94% | 1287.5 ± 0.5 | 1288.0 |
| | GalNAc6S-GlcA-pNP | | | | |
| 6 | GlcA-GalNH₂-GlcA- | 20 | 90% | 1031.5 ± 0.6 | 1031.8 |
| | GalNAc-GlcA-pNP | | | | |
| 7 | GlcA-GalNH₂6S-GlcA- | 4 | 96% | 1191.8 ± 0.6 | 1191.2 |
| | GalNAc6S-GlcA-pNP | | | | |
| 8 | GlcA-GalN_{Az}-GlcA- | 5 | >98% | 1057.6 ± 0.7 | 1057.8 |
| | GalNAc-GlcA-pNP | | | | |
| 9 | GlcA-GalNH_{Az}6S-GlcA- | 5 | >98% | 1217.6 ± 0.6 | 1217.9 |
| | GalNAc6S-GlcA-pNP | | | | |
| 10 | GlcA-GalNAc6S-GlcA- | 30 | >98% | 1693.8 ± 1.1 | 1693.4 |
| | GalNAc6S-GlcA- | | | | |
| | GalNAc6S-GlcA-pNP | | | | |
| 11 | GlcA-GalNAc-GlcA- | 11 | 94% | 1613.3 ± 0.6 | 1613.3 |
| | GalNAc4S-GlcA- | | | | |
| | GalNAc4S-GlcA-pNP | | | | |
| 12 | GlcA-GalNAc-GlcA- | 5 | >98% | 1533.2 ± 0.5 | 1533.2 |
| | GalNAc-GlcA-GalNAc6S- | | | | |
| | GlcA-pNP | | | | |
| 13 | GlcA-GalNAc-GlcA- | 19 | >98% | 1613.3 ± 0.6 | 1613.3 |
| | GalNAc6S-GlcA- | | | | |
| | GalNAc6S-GlcA-pNP | | | | |
| 14 | GlcA-GalNAc6S-GlcA- | 22 | >98% | 2153.5 ± 1.1 | 2152.7 |
| | GalNAc6S-GlcA- | | | | |
| | GalNAc6S-GlcA- | | | | |
| | GalNAc6S-GlcA-pNP | | | | |
| 15 | GlcA-GalNAc-GlcA- | 14 | >98% | 2073.6 ± 1.2 | 2072.7 |
| | GalNAc4S-GlcA- | | | | |
| | GalNAc4S-GlcA- | | | | |
| | GalNAc4S-GlcA-pNP | | | | |

¹Purity was determined by high resolution anion exchange HPLC analysis.
²The product is a mixture of compound 4 and its detrifluoroacetylated product as demonstrated by ESI-MS analysis.

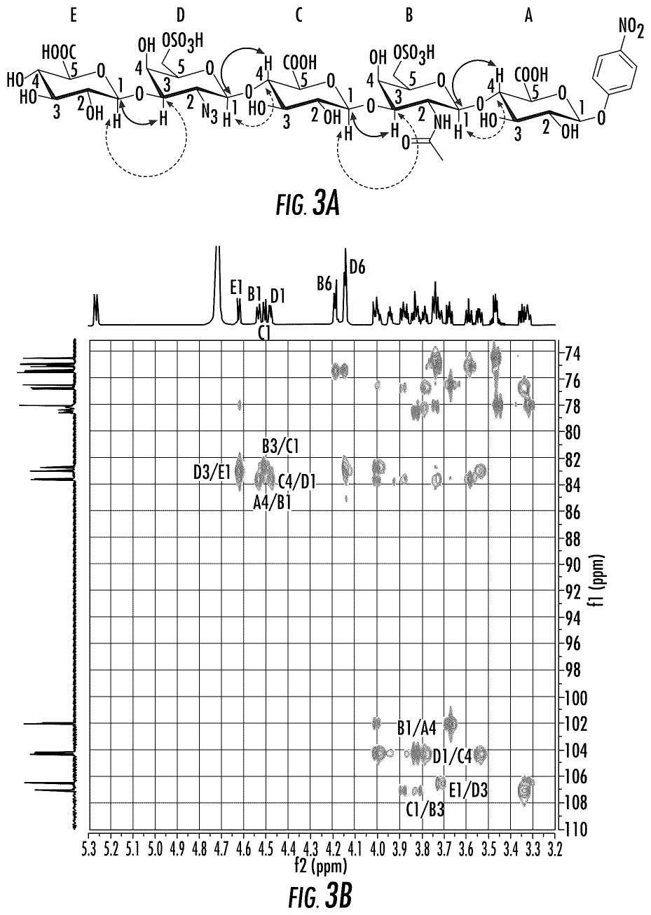

The purity and structure of products were characterized by high resolution anion-exchange HPLC, electrospray ionization mass spectrometry (ESI-MS) and NMR. The majority of products displayed a single symmetric peak on HPLC, confirming the purities (Table 1). The structure of each compound was confirmed by 1D-and 2D-NMR with full assignments. One representative example for the structural analysis of compound 9 using heteronucluear multiple-bond correlation spectroscopy (HMBC) of 2D-NMR is shown in FIG. 3B. Cross signal of residue anomeric proton of residue C and carbon (C-4) of residue B was observed (dashed double arrows in FIG. 3A); likewise cross signal of the proton (H-4) of residue B and anomeric carbon of residue C was also observed (solid double arrows in FIG. 3A). The cross peak signals suggest that the glycosidic linkage is 1→3 link. Using a similar approach, it was revealed that the glycosidic linkage between residue E and D is also 1→3 link. The anomeric proton-proton coupling constant (³J_{H-H}) values of residue B, C, D and E were determined to be 8.5, 7.9, 8.2 and 7.9 Hz, respectively, from 1D-NMR analysis. The measured ³J_{H-H} values suggest that the glycosidic linkages in compound 9 are in the [symbol] -form. Comparing the chemical shifts of H-6 from residues D and B in compound 9 and compound 8 revealed the presence of 6-O-sulfation in compound 9. The chemical shifts of H-6 were changed to 4.19 ppm (residue B, compound 9) from 3.72-3.74 ppm (residue B, compound 8), and 4.14-4.15 ppm (residue D, compound 9) from 3.68-3.72 ppm (residue D, compound 8).

Thus, in some embodiments provided herein are novel methods to synthesize structurally homogeneous 4-O-sulfated and 6-O-sulfated CS oligosaccharides. The enzymatic approach is highly divergent to prepare a range of oligosaccharides merely using a small number of enzymes and cofactors. These methods, as shown herein, can be suitable for the preparation of structurally diversified CS oligosaccharide libraries. Prior to the instant disclosure the enzymatic synthesis of CS was perceived as unable to prepare a sufficiently large quantity of structurally defined CS oligosaccharides for biological evaluations and studies. By way of example and not limitation, the disclosed enzymatic methods of synthesizing CS can yield milligram to gram quantities, ranging from about 4 milligrams to about 40 milligrams, or more. In some aspects, the disclosed synthetic routes can yield at least about 30 milligrams or more. In some aspects, the disclosed synthetic routes can yield industrially applicable quantities, including for example at least about 1 gram or more, or about 1 gram to about 100 grams or more.

In development of the disclosed methods of CS synthesis significant challenges were overcome, including for example improving the accessibility of recombinant CS biosynthetic enzymes and reducing the production cost for UDP-GalNAc. In addition to synthesizing natural CS oligosaccharides, the instant disclosure provides for the possibility of preparing unnatural 6-O-sulfated CS oligosaccharides. The synthesis was accomplished in multi-milligram scales, allowing complete structural characterization by MS and NMR. In addition to CS-A and CS-C, CS-D and CS-E were also synthesized by methods disclosed herein. The demonstration of the enzymatic synthesis of structurally defined CS oligosaccharides offers an essential tool to investigate the biological functions of CS.

In addition to the CS compounds ranging from trisaccharides to nonasaccharides, i.e. 9-mers, as shown in Table 1 and FIG. 1, the disclosed methods provide for the synthesis of larger and/or longer CS compounds, as shown in FIGS. 4, 5A, 5B, 6A, 6B, 7A, 7B, and 8, and below.

Example CS-A oligosaccharides (11-mer to 19-mer):

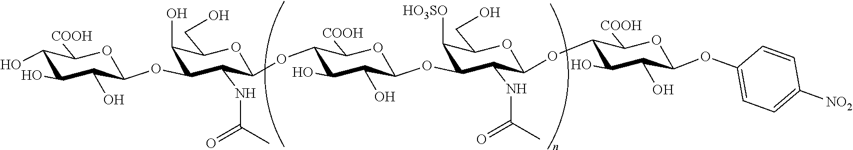

n=4, CS-A 11-mer
n=5, CS-A 13-mer
n=6, CS-A 15-mer
n=7, CS-A 17-mer
n=8, CS-A 19-mer

Example CS-C oligosaccharides (11-mer to 19-mer):

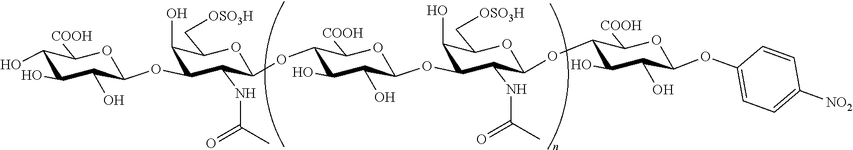

n=4, CS-C 11-mer
n=5, CS-C 13-mer
n=6, CS-C 15-mer
n=7, CS-C 17-mer
n=8, CS-C 19-mer

Example CS-E oligosaccharides (5-mer to 19-mer):

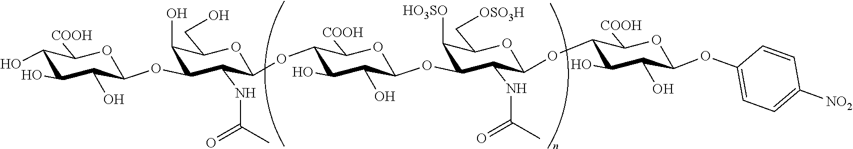

n=1, CS-E 5-mer
n=2, CS-E 7-mer
n=3, CS-E 9-mer
n=4, CS-E 11-mer
n=5, CS-E 13-mer
n=6, CS-E 15-mer
n=7, CS-E 17-mer
n=8, CS-E 19-mer

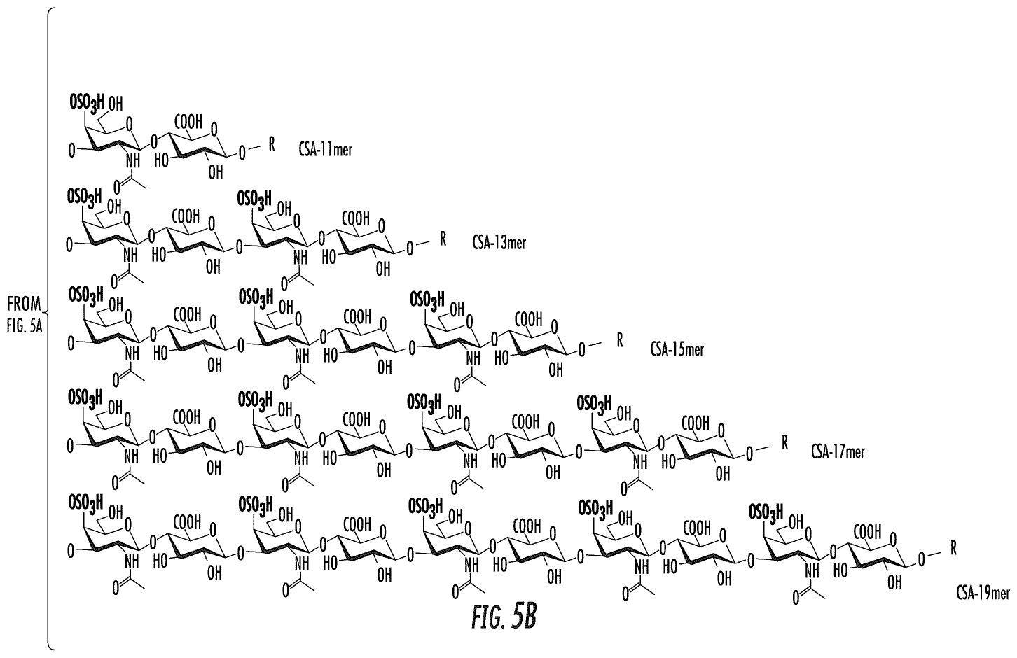

FIG. 4 also shows a plurality of CS-A, CS-C and CS-E compounds synthesized by the disclosed methods and schematics. These CS compounds are shown in an abbreviated form in FIG. 4, with the full CS structures shown in FIGS. 5A, 5B, 6A, 6B, 7A and 7B.

Approaches for synthesizing CS-D and CS-E were developed as disclosed herein. Such methods utilize similar approaches as with the synthesis of CS-A and CS-C, but with additional enzymatic steps. For example, in some embodiments the synthesis of CS-D and/or CS-E requires additional CS sulfotransferases, including for example CS 2-O-sulfotransferase (to prepare CS-D) and GalNAc4S-6-O-sulfotransferase (to prepare CS-E). The instant disclosure provides both 2-O-sulfotransferase and GalNAc4S-6-O-sulfotransferase needed for such additional steps. By way of example and not limitation, FIG. 8 illustrates a method of synthesizing CS-E from CS-A to yield CS-E compounds ranging from 5-mer to 19-mer. In such methods, for example, GalNAc4S6OST can convert a CS-A compound to a CS-E compound.

Thus, in some embodiments, disclosed herein are methods of synthesizing chondroitin sulfate oligosaccharides. Such methods can comprise in some embodiments providing a chondroitin backbone and completing at least two, or three, or four of the following enzymatic reaction steps in FIG. 1, including for example an elongation step to add a GalNAc residue using a glycosyltransferase from E. coli K4 (KfoC), an elongation step to add a GlcA residue using KfoC, a 6-O-sulfation step using chondroitin sulfate 6-O-sulfotransferase (CS6OST) and sulfate donor 3′-phosphoadenosine 5′-phosphosulfate (PAPS), and/or a 4-O-sulfation step involving chondroitin sulfate 4-O-sulfotransferase (CS4OST) and PAPS. Such methods can in some aspects further comprise a step of transferring a GalNTFA residue to a trisaccharide backbone by a KfoC to yield a tetrasaccharide. In some aspects each of these reaction steps can be performed more than once, as illustrated in FIGS. 1, 2A, and 2B.

Such methods can yield substantially homogenous chondroitin sulfate oligosaccharides, including for example 4-O-sulfated and/or 6-O-sulfated chondroitin oligosaccharides having a size ranging from a trisaccharide to a nonasaccharide. In some aspects the synthesized chondroitin sulfates can have a size ranging from a 3-mer to a 15-mer, or more. Such methods can also yield unnatural chondroitin sulfate oligosaccharides. Such methods can also yield structurally defined chondroitin sulfate oligosaccharide, in some aspects in multi-milligram quantities. In some aspects, such synthetic chondroitin sulfate oligosaccharides can include chondroitin sulfate A (CS-A), chondroitin sulfate C (CS-C), chondroitin sulfate D (CS-D) and chondroitin sulfate E (CS-E), including those depicted in FIGS. 1, 4, 5A, 5B, 6A, 6B, 7A, 7B, and 8.

In FIGS. 1, 2A, 2B, 5A, 5B, 6A, 6B, 7A, 7B, and 8, the R group can in some embodiments be selected from the group consisting of —H, alkyl (such as but not limited to —CH₃ or —CH₂CH₃), substituted alkyl, aryl, and substituted aryl (such as but not limited to a p-nitrophenyl group). In some embodiments, the R group can include a pNP compound as shown, for example, in FIGS. 3 and 4, and hereinabove. However, the illustration of the compounds hereinabove and in FIGS. 3 and 4 with the pNP constituent is for illustrative purposes only, as such compounds can alternatively include an R group as defined hereinabove.

### Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

A structure represented generally by a formula such as:

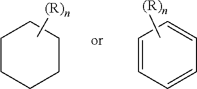

as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

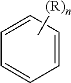

wherein n is an integer from 0 to 2 comprises compound groups including, but not limited to:

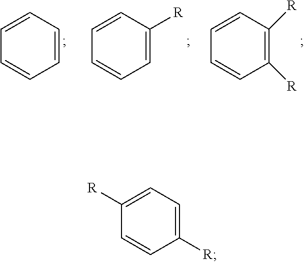

and the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The term “heterocycle” refers to a non-aromatic or aromatic, monocyclic or multicyclic ring system of about 3 to about 14 atoms, wherein at least one of the atoms is a heteroatom (e.g., oxygen, nitrogen, or sulfur). The term “N-heterocycle” refers to a heterocycle wherein at least one of the heteroatoms is a nitrogen atom. Examples of N-heterocycles include, but are not limited to, azetidine, pyrrolidine, pyrrole, pyrroline, piperidine, pyridine, piperazine, pyrazine, pyrimidine, pyridazine, morpholine, and thiazine.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

As used herein, the term “acyl” refers to an organic carboxylic acid group wherein the —OH of the carboxyl group has been replaced with another substituent (i.e., as represented by RC(═O)—, wherein R is an alkyl, substituted alkyl, aralkyl, aryl or substituted aryl group as defined herein). As such, the term “acyl” specifically includes arylacyl groups, such as an acetylfuran and a phenacyl group. Specific examples of acyl groups include acetyl and benzoyl.

“N-acyl” refers to a group having the structure —N—C(═O)—R, wherein R is as defined for acyl. These groups can also be referred to as amides. Modified N-acyl groups include compounds wherein the oxygen of the N-acyl has been replaced by S or NH, as well as to compounds wherein the carbonyl group (i.e., the —C(═O)—) is attached to a second heteroatom in addition to the nitrogen. For example, the carbonyl can be attached to a second nitrogen atom to form a urea linkage (i.e., —NH—C(═O)—NH—R).

The term “amino” refers to the —NH₂, the —NHR, and the —NR₂ groups, wherein each R is independently alkyl, substituted alkyl, aryl, substituted aryl, or aralkyl, as well as to amino and ammonium functionalities in N-heterocycles (e.g., morpholine, etc). As used herein the term “amino” can also refer to substituents that provide quaternary ammonium cations, such as —⁺NH₃, —⁺NH(R)₂, and —⁺N(R)₃ groups, wherein each R is independently alkyl, substituted alkyl, aryl, substituted aryl or aralkyl.

The term “ester” refers to a moiety comprising an —O—C(═O)—R group, wherein R can be alkyl, substituted alkyl, aralkyl, aryl, or substituted aryl. In some embodiments, the R group can include an amino substituent and the ester is an amino ester.

The term “amide” refers to a moiety comprising a —N(R′)—C(═O)—R group, wherein R is selected from alkyl, substituted alkyl, aralkyl, aryl or substituted aryl and R′ is H, alkyl, substituted alkyl, aralkyl, aryl, or substituted aryl.

The term “urea” as used herein can refer to a moiety comprising a —N(R′)—C(═O)—N(R′)-group, wherein each R′ is independently H, alkyl, substituted alkyl, aralkyl, aryl, or substituted aryl.

The term “hydroxyl” refers to the —OH group.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like.

### EXAMPLES

The following Examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### EXPERIMENTAL PROCEDURES

### Construction of Expression Plasmids for KfoC, AGX1, CS6OST and CS4OST

Both KfoC (coding the chondroitin polymerase) and agxl (coding the UDP-N-acteylglucosamine pyrophosphorylase 1) were cloned into pET15b vector (Novagen) to construct Nterminal (His)6-tagged proteins. The 2.1 kb DNA containing the whole kfoC gene was amplified from E. coli K4 genome using primer pair KfoC-F and KfoC-R. Phusion high fidelity DNA polymerase (NEB) was used to keep the fidelity. The PCR product was cloned into the Ndel/BamHI sites using HiFi DNA assembly master mix (NEB) to give the expression plasmid pET15b-kfoC. Sequencing was applied to ensure the correctness of the gene (Eurofins Genomics). Using the similar strategy, the 1.5 kb agx1 gene was amplified from human cDNA clone UAP1 (Origene) using primer pair AGX1-F and Agx1-R. The DNA fragment was cloned into pET15b Ndel/BamHI sites using the same method as cloning kfoC. The constructed agx1 expression plasmid pET15b-agx1 was also subjected to sequencing to make sure the gene is correct.

To express the chondroitin sulfotransferase, the genes responsible for human CS4OST and mouse CS6OST were cloned into pFastBac-Mel-HT vector. For CS4OST, primer pair hCS-4-OST-1-F and hCS-4-OST-1-R were used to PCR amplify the CS4OST coding gene from cDNA clone IMAGE 1283775. The 1.0 kb PCR product was cloned into pFastBac-Mel-HT EcoRI/Xbal sites using HiFi DNA assembly master mix (NEB) to generate the recombinant plasmid pFastBa-Mel-HT-CS4OST. For CS6OST, PCR was also applied with mCS-6-OST-F and mCS-6-OSTR as the primer pair, cDNA clone IMAGE 6493234 as the template. The resultant 1.3 kb PCR product was inserted into the EcoRI/Xbal sites of pFastBac-Mel-HT using the same strategy for cloning CS4OST coding gene to afford plasmid pFastBac-Mel-HT-CS6OST.

### Expression of Recombinant KfoC, AGX1, CS4OST and CS6OST

The expression of KfoC and Agxl were performed in E. coli BL21 cells. The expression plasmids pET15bkfoC and pET15b-agx1 were transformed into E. coli BL21 competent cells (Invitrogen). The transformed cells were grown in LB medium containing 50 μg/L kanamycin and incubated at 37° C. until the OD600 reached 0.6-0.8. Then a final concentration of 0.2 mM isopropylthiogalactopyranoside (IPTG) was added to induce the expression of the proteins. The cultures were kept at 22° C. and 195 rpm overnight. The E. coli cells were harvested by centrifuge with 6,000 rpm for 10 min, then resuspended in 25 mM Tris, 30 mM imidazole, and 500 mM NaCl, pH 7.5. Sonication was used to lysis the cell, and subsequent centrifuge was at 14,000 rpm for 30 min. The supernatant was subjected to a nickel agarose column (GE Healthcare). Buffer A (25 mM Tris, 60 mM imidazole, and 500 mM NaCl, pH 7.5) was used to wash the column and buffer B (25 mM Tris, 250 mM imidazole, 500 mM NaCl, pH 7.5) was used to elute the proteins. All the proteins were kept in 20% glycerol at −80° C.

The expression of CS6OST and CS4OST was carried out in the Sf9 cells (Invitrogen) using Sf-900™ III SFM media (Life Technologies). The sequencing correct plasmids (pFastBac-Mel-HT-CS6OST and pFastBac-Mel-HT-CS4OST) were transformed into E. coli DH10Bac cells (Invitrogen). The recombinant viruses for expression of CS6OST and CS4OST were made following the instruction manual (Invitrogen). Infection of the recombinant virus for expression of CS6OST and CS4OST was carried out when the insect cells were at a concentration of 2.0×106 cells/mL. The culture was incubated in the shaker at 27° C. for another 4 to 5 days. Culture was harvested by centrifuge (4,000 rpm, 10 min) and the supernatant was added 1 mM phenylmethanesulfonyl fluoride (PMSF), 0.1% Triton X-100 and 2% glycerol, adjusted the pH to 7.0 by NaOH solution. Another centrifuge (8,000 rpm, 30 min) was applied to remove the precipitates. The supernatant culture was then mixed with an equal volume of 20 mM 3-(N-morpholino) propanesulfonic acid (MOPS) buffer containing 0.1% triton X-100, 2% Glycerol pH 7.0) filtered, and loaded to a heparin toyopearl (Tosoh Bioscience) column. Buffer C (20 mM MOPS, 100 mM NaCl, 2% glycerol and 0.1% reduced triton X-100 (Sigma), pH 7.0) was used to wash the column until UV absorbance at 280 nm reached baseline. Gradient elution of 0-100% Buffer D (20 mM MOPS, 1 M NaCl, 2% glycerol and 0.1% reduced triton X-100, pH 7.0) was used to elute the target proteins. The collections with chondroitin sulfotransferase activity were collected. To further purify the chondroitin sulfotransferases and remove the chondroitinase produced by the insect cells, the collected proteins were then dialyzed by the nickel column buffer A and then load to a cobalt (for CS4OST) or a nickel column (for CS6OST). A gradient elution of 0-100% nickel column buffer B was applied to elute the proteins. The collected proteins with chondroitin sulfotransferase activity were kept in 20% glycerol at −80° C.

### Measurement of the Activities of CS4OST and CS6OST

Chondroitin sulfotransferase activity of the proteins was determined by incubating 2-5 μl purified proteins with 5 μg chondroitin (Seikagaku) and 1-5×105 cpm of [35S]PAPS (5 μM) in 100 μl buffer containing 50 mM MOPS (pH 7.0). The reaction was incubated at 37° C. for 2 hours and quenched by adding 400 μl UPAS buffer (50 mM NaAcO, 150 mM NaCl, 4 M Urea, 1 mM EDTA and 0.1% Triton X-100, pH5.5). Then the samples were loaded to 200 μl DEAE-sepharose columns and washed by 4 ml UPAS buffer, 4 ml washing buffer (50 mM NaAcO, 250 mM NaCl and 0.1% Triton X-100, pH5.5). The [35S]-chondroitin was eluted by the elution buffer (50 mM NaAcO, 1 M NaCl and 0.1% Triton X-100, pH5.5) and subjected to liquid scintillation counting.

### Measurement of the Activity of Chondroitinase

It might be desirable to measure the level of contaminated viral chondroitinase in the recombinant CS4OST and CS6OST preparations. Briefly, 0.1 mg chondroitin was incubated with 5 μl culture or purified enzyme in a total of 50 μl a buffer containing 50 mM MOPS (pH7.0) at 37° C. overnight. Then the reaction mixture was diluted 10 times and tested the absorbance at the UV 232 nm.

### Preparation UDP-GalNAc and UDP-GalNATFA

To synthesis UDP-N-acetylgalactosamine (UDP-GalNAc), one of the chondroitin biosynthesis cofactors, the substrate N-acetyl-D-galactosamine (GalNAc, Carbosynth) 4 mmol was incubated with 5 mMol ATP, 4.5 mmol UTP, 45 mg NAHK, 36 mg AGXI and 4.2 mg pyrophosphatase in a buffer containing 50 mM Tris-HCl buffer (pH 7.5), 10 mM MgSO4 in a total volume of 200 mL. The reaction mixture was incubated at 37° C. overnight and the product UDPPage GalNAc was monitored by HPLC and ESI-MS. The synthesis of UDP-GalNTFA, 3.6 mmol GalNTFA, was incubated in the same reaction system and conditions used for synthesis of UDP-GalNAc. GalNTFA is prepared according to the literature.

### Conversion of GalNTFA to GalNH2 and GalNAZ

To obtain compound 4 with a GalNTFA unit in the chondroitin backbone, the synthesis was initiated from chondroitin trisaccharide. The conversion of chondroitin trisaccharide to compound 4 involved 2 steps elongation. In summary, 100 mg trisaccharide backbone was incubated with 0.3 mmol UDP-GalNTFA, 25 mg KfoC in the same buffer as that used for elongation of the chondroitin backbone. The resultant tetrasaccharide with a GalNTFA unit at the nonreducing end was purified by a C18 column (Biotage) and then used as the substrate for compound 4. At this step, the GlcA residue was added at the presence of KfoC, in the elongation buffer as mentioned before. Compound 4 was also purified by a C18 column at the same condition used for purification of chondroitin backbone. To get rid of the salt in the solution, compound 4 was further purified by a P2 column.

To convert the GalNTFA residue to GalNH2 residue, LiOH solution was added to compound 4 solution (about 1 mg/ml) in a total volume of 50 ml to reach the final concentration of 0.1 M. HPLC with a Polyamine II column and MS were used to monitor the degree of the detrifluoroacetylation reaction. Upon the completion of detrifluoroacetylation, the pH of the reaction mixture was adjusted to 2.0 for purification by a C18 column. Then compound 6 was further purified by a P2 column.

Synthesis of compound 8 was completed from compound 6. To a solution of compound 6 in water (2 mL, 10 mg/mL), MeOH (20 mL) was added, along with 0.5 mL of a Cu(II)SO4·5H2O solution in water (1 mg/mL) and Et3N (50 μL). After mixing, Imidazole-1-sulfonyl azide hydrochloride (prepared according to the literature 3) was added (41 mg, 10 equiv) and the reaction was left agitated for 48 h. The produced compound 8 was subjected to a Giga Q chromatography (Tosoh Bioscience) and then a BioGel P2 column (BioRad) for purification.

### EXAMPLE 1

### Synthesis of Chondroitin Backbone

Compound 1 was synthesized from chondroitin trisaccharide backbone. Briefly, 10 mg trisaccharide was incubated with 0.1 mmol 3′-phosphoadenosine 5′-phosphosulfate (PAPS) in 100 ml buffer containing 50 mM MOPS (pH 7.0) and 10 ml purified CS6OST, 37° C. overnight. HPLC equipped with Polyamine II column (YMC) was used to monitor the reaction. Then, the product compound 1 was subjected to a column with Toyopearl GigaCap Q-650 resin (Tosoh Bioscience) for purification. The elution of the Giga Q column was using a gradient of 0-100% buffer B (1M NaCl, 20 mM Tris-HCl, pH 7.0) from Buffer A (20 mM Tris-HCl, pH 7.0) in 120 minutes, at the flow rate of 0.7 ml/min. The collected compound 1 was then dried, dissolved in 2 ml H2O and subjected to a semi-preparative polyamine II column (250×4.6 mm, S-5 μm, YMC). The elution condition was used as follows: the gradient elution was 40-100% KH2PO4 (1M) buffer in 40 min at flow rate 1 ml/min. The collection was further purified by a Bio-Gel P2 column using 0.1 M ammonium bicarbonate (Sigma) at a flow rate of 4 mL/h. The resultant compound 1 was then analyzed by MS and NMR spectra.

### EXAMPLE 2

### Synthesis of CS Compounds

The synthesis of compound 2 was started from chondroitin pentasaccharide. Briefly, 39 mg pentasaccharide backbone was incubated with 0.24 mmol PAPS, 10 mL purified CS6OST in a total 100 mL volume containing 50 mM MOPS (pH 6.8), 37° C. overnight. The completion of the reaction was monitored by injecting a small amount of reaction mixture to HPLC using Polyamine II column. If reaction was not complete, additional CS6OST enzyme and PAPS was added, and kept the reaction at 37° C. for another day. The product compound 2 was purified by Giga™ Q column and P2 column subsequently using the same conditions as those for purification of compound 1. Then compound 2 was subjected to MS and NMR analysis.

Compound 3 was synthesized from pentasaccharide backbone. Briefly, 20 mg chondroitin pentasaccharide was incubated with 0.07 mmol PAPS, 7 ml CS4OST in a total 200 ml buffer containing 50 mM MES (pH 6.5), 2 mM dithiothreitol (DTT) and 20 mM CaCl2 at 37° C. overnight. The synthesized compound 3 was purified sequentially by a Giga™ Q column, a semi-preparative polyamine II column and a BioGel P2 column.

Compound 5 has C-6 sulfation of the GalNAc residues of compound 4. Here, 6 mg compound 4 was incubated with 0.03 mmol PAPS and 5 ml CS6OST in 50 ml reaction buffer containing 50 mM MOPS (pH 6.8). After incubation at 37° C. overnight, the product was purified by a Giga™ Q column and subsequently a P2 column, following the conditions mentioned before.

To obtain compound 7, 10 mg compound 6 was incubated with 0.05 mmol PAPS in a reaction mixture containing 10 ml CS6OST, 50 mM MOPS (pH 6.8), at 37° C. for 2 days. The resultant compound 7 was purified by a Giga Q column, a semi-preparative polyamine II column and a subsequent P2 column, as conditions for purification of compound 1.

To obtain compound 9, compound 8 (about 7 mg) was further sulfated by 5 ml CS-6OST in the 50 ml reaction mixture containing 0.03 mmol PAPS and 50 mM MOPS (pH 6.8). The reaction mixture was incubated at 37° C. overnight, and the resultant compound 9 was purified by a Giga Q column and a subsequent P2 column.

To obtain compound 10, 29 mg chondroitin heptasaccharide was incubated with 0.24 mmol PAPS, 5 ml CS-6OST in 100 ml buffer containing 50 mM MOPS (pH 6.8) at 37° C. overnight. The HPLC results showed that the reaction was not complete, so another 4 ml CS-6OST was added into the reaction mixture. The reaction was incubated at 37° C. for another day until the reaction was almost complete. The purification of compound 10 was subjected to a Giga Q column with a gradient elution of 20-100% buffer B (1M NaCl, 20 mM sodium acetate, pH 5.0) from Buffer A (20 mM sodium acetate, pH 5.0) in 120 minutes, at the flow rate of 1 ml/min. The collected product was then dried and dialyzed to remove the salt for MS and NMR analysis.

To obtain compound 11, 18 mg chondroitin heptasaccharide backbone was incubated with 0.1 mmol PAPS, 7 ml CS4OST in 200 ml buffer containing 50 mM MES (pH 6.5), 2 mM DTT and 20 mM CaCl2 at 37° C. overnight. The produced compound 11 was purified by a Giga Q column under the same condition for compound 10 and then dialyzed for MS and NMR analysis.

The synthesis of compound 12 from compound 1 involves 4 steps elongation, including alternative add GalNAc and GlcA units two times to the non-reducing end. The product after each step was analyzed by HPLC, MS and purified by a Giga Q column following the condition used for purification of compound 10. The final product compound 12 was further subjected to dialysis to remove the salt.

The conversion of compound 2 to 13 involved 2 steps elongation to add GalNAc and GlcA residues to the non-reducing end of compound 2 using the same conditions as those for synthesis of chondroitin backbone. After each step, the products was purified by Giga Q chromatography as compound 10 and desalted by dialysis if it was needed.

To generate compound 14, 22 mg nanosaccharide was incubated with 0.16 mmol PAPS, 15 ml CS-6OST in a total 100 ml volume containing 50 mM MOPS (pH 6.8), 37° C. overnight. The product compound 14 was purified by Giga Q column and then subjected to dialysis for desalination.

The synthesis of compound 15 was initiated from 20 mg chondroitin nonasaccharide backbone. It was incubated with 0.1 mmol PAPS, 7 ml CS4OST, 50 mM MES (pH 6.5), 2 mM DTT and 20 mM CaCl2 in a total volume of 200 mL. The reaction mixture was kept at 37° C. overnight. The product was subjected to a Giga™ Q chromatography and subsequent dialysis.

### EXAMPLE 3

### HPLC Analysis of CS Compounds

HPLC analysis of the chondroitin compounds was carried out via a Polyamine II column. Gradient elution was applied for elution. For the chondroitin backbones, the concentration of KH2PO4 buffer (1M) was increased from 0 to 50% in 20 min at flow rate 0.5 ml/min. The detect UV was set at 310 and 260 nm. For the chondroitin A and chondroitin C oligosaccharides, the concentration of KH2PO4 buffer (1M) was increased from 0 to 100% in 20 min at flow rate 1 ml/min. The oligosaccharides were detected at 310 nm absorption.

### EXAMPLE 4

### ESI-MS Analysis of CS Compounds

The analyses were performed at a Thermo LCQ-Deca. CS oligosaccharides were directly diluted in 500 μl H2O. A syringe pump (Harvard Apparatus) was used to introduce the sample by direct infusion (50 μl min−1). Experiments were carried out in negative ionization mode. The electrospray source was set to 3 KV and 150° C. The automatic gain control was set to 1×107 for full scan MS. The MS data were acquired and processed using Xcalibur 1.3.

### EXAMPLE 5

### NMR Analysis of CS Compounds

NMR experiments were performed at 298 K on Bruker Avance™ 700 MHZ and 850 MHz spectrometer with Topsin™ 3.2 software. Samples (0.5 to 3.0 mg) were each dissolved in 0.5 ml D20 (99.996%, Sigma-Aldrich) and lyophilized three times to remove the exchangeable protons. The samples were re-dissolved in 0.5 ml D2O and transferred to NMR microtubes (O.D. 5 mm, Norrell). Chemical shifts are referenced to external 2,2-dimethyl-2-silapentane-5-sulfonate sodium salt (DSS, Sigma, Co.). 1D 1HNMR experiments “zg” pulse sequence were performed with 64 scans and an acquisition time of 3.8 sec. 1D 13C-NMR experiments “zgdc30” pulse sequence were performed with 10,000 scans and an acquisition time of 1.0 sec. 2D 1H-13C HSQC experiments “hsqcgpph” pulse sequence were performed with 48 scans, 512 increments, 1.5 sec relaxation delay, and 120 msec acquisition time. 2D spectra were recorded with GARP carbon decoupling. 48 dummy scans were used prior to the start of acquisition. 2048 total points were collected in f2. 13C transmitter offset was set at 90.0 ppm.

### REFERENCES

ADDIN EN.REFLIST
1. Mizumoto, S.; Yamada, S.; Sugahara, K., Curr. Opin. Struct. Biol. 2015, 34, 35-42.
2. Fried, M. W.; Duffy, P. E., Vaccine 2015, 33, 7483-7488.
3. Chan, S.; Frasch, A.; Mandava, C. S.; Ch'ng, J.-H .; Quintana, M. d. P.; Vesterlund, M.; Ghorbal, M.; Joannin, N.; Franzén, O.; Lopez-Rubio, J.-J.; Barbieri, S.; Lanzavecchia, A.; Sanyal, S.; Wahlgren, M., Nature Microbiology 2017, 2, 17068.
4. Bradbury, E. J.; Moon, L. D.; Popat, R. J.; King, V. R.; Bennett, G. S.; Patel, P. N.; Fawcett, J. W.; McMahon, S. B., Nature 2002, 416, 636-640.
5. Kyiyata, S.; Komatsu, Y.; Yoshimura, Y.; Taya, C.; H, K., Nat. Neurosci. 2012, 15, 414-422.
6. Brown, J. M.; Xia, J.; Zhuang, B.; Cho, K .-S.; Rogers, C. J.; Gama, C. I.; Rawat, M.; Tully, S. E.; Uetani, N.; Mason, D. E.; Tremblay, M. L.; Peters, E. C.; Habuchi, O.; Chen, D. F.; Hsieh-Wilson, L. C., Proc. Natl. Acad. Sci. U S A 2012, 109, 4768-4773.
7. Gama, C.; Tully, S. E.; Sotogaku, N.; Clark, P. M.; Rawat, M.; Vaidehi, N.; Goddard, W. A.; Nishi, A.; Hsieh-Wilson, L. C., Nat Chem Biol 2006, 2, 467-473.
8. Yang, X.; Lin, Y. P.; Heselpoth, R. D.; Buyuktanir, O.; Qin, J.; Kung, F.; Nelson, D. C.; Leong, J. M.; Pal, U., Cell Microbiol 2016, 18, 97-110.
9. Pulsipher, A.; Griffin, M. E.; Stone, S. E.; Brown, J. M.; Hsieh-Wilson, L. C., J. Am. Chem. Soc. 2014, 136, 6794-6797.
10. Stabler, T. V.; Huang, Z.; Montell, E.; Verges, J.; Kraus, V. B., Osteoarthritis and Cartilage 2017, 25, 166-174.
11. Roman-Blas, J. A.; Mediero, A.; Tardio, L.; Portal-Nunez, S.; Gratal, P.; Herrero-Beaumont, G.; Largo, R., Eur. J. Pharmacol. 2017, 794, 8-14.
12. Ly, M.; Leach III, F. E.; Laremore, T. N.; Toida, T.; Amster, I. J.; Linhardt , R. J., Nat. Chem. Biol. 2011, 7, 827-833.
13. Tamura, J .-i.; Nakada, Y.; Taniguchi, K.; Yamane, M., Carbohydr. Res. 2008, 343, 39-47.
14. Lopin-Bon, C.; Jacquinet, J .-C., Carbohydr. Res. 2015, 402, 35-43.
15. Miyachi, K.; Wakao, M.; Suda, Y., Bioorg Med Chem Lett 2015, 25, 1552-1555.
16. Solera, C.; Macchione, G.; Maza, S.; Kayser, M. M.; Corzana, F.; de Paz, J. L.; Nieto, P. M., Chemistry 2016, 22, 2356-2369.
17. Jacquinet, J .-C.; Lopin, C., Angew. Chem. Int. Ed. 2006, 45, 2574-2578.
18. Eller, S.; Collot, M.; Yin, J.; Hahm, H. S.; Seeberger, P. H., Angew. Chem. Int. ed. 2013, 52, 5858-5861.
19. Sugiura, N.; Shioiri, T.; Chiba, M.; Narimatsu, H.; Kimata, K.; Watanabe, H., J. Biol. Chem. 2012, 287, 43390-43400.
20. Sugiura, N.; Clausen, T. M.; Shioiri, T.; Gustavsson, T.; Watanabe, H.; Salanti, A., Glycoconj J 2016, in press, DOI 10.1007/s10719-016-9685-z.
21. Shioiri, T.; Tsuchimotot, J.; Watanabe, H.; Sugiura, N., Glycobiology 2016, 26, 592-606.
22. Sugiura, N.; Shimokata, S.; Minamisawa, T.; Hirabayashi, J.; Kimata, K.; Watanabe, H., Glycoconj J 2008, 25, 521-530.
23. Xue, J.; Jin, L.; Zhang, X.; Wang, F.; Ling, P.; Sheng, J., Biochim Biophys Acta 2016, 1860, 844-855.
24. Zhao, G.; Guan, W.; Cai, L.; Wang, P. G., Nat. Protoc. 2010, 5, 636-646.
25. Bouregeaux, V.; Piller, F.; Piller, V., Bioorg Med Chem Lett 2005, 15, 5459-5462.
26. Sugiura, N.; Setoyama, y.; Chiba, M.; Kimata, K.; Watanabe, H., J. Biol. Chem. 2011, 286, 29026-29034.
27. Yusa, A.; Kitajima, K.; Habuchi, O., J. Biol. Chem. 2006, 281, 20393-20403.

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of synthesizing a synthetic chondroitin sulfate oligosaccharide, the method comprising:
providing a chondroitin backbone; and
performing at least two of the following enzymatic reaction steps:
an elongation step to add a GalNAc residue using a glycosyltransferase from E. coli K4 (KfoC);
an elongation step to add a GlcA residue using KfoC;
a 6-O-sulfation step using chondroitin sulfate 6-O-sulfotransferase (CS6OST) and sulfate donor 3′-phosphoadenosine 5′-phosphosulfate (PAPS); and
a 4-O-sulfation step involving chondroitin sulfate 4-O-sulfotransferase (CS4OST) and PAPS;
whereby a synthetic chondroitin sulfate oligosaccharide is synthesized.

2. The method of claim 1, further comprising a step of transferring a GalNTFA residue to a trisaccharide backbone by a KfoC to yield a tetrasaccharide.

3. The method of any of claims 1 to 2, wherein at least three of the enzymatic reaction steps are performed.

4. The method of any of claims 1 to 3, wherein at least four of the enzymatic reaction steps are performed.

5. The method of any of claims 1 to 4, wherein the method yields substantially homogenous chondroitin sulfate oligosaccharides.

6. The method of any of claims 1 to 5, wherein the method yields 4-O-sulfated and/or 6-O-sulfated chondroitin oligosaccharides having a size ranging from a trisaccharide to a nonasaccharide.

7. The method of any of claims 1 to 5, wherein the method yields a chondroitin sulfate ranging in size from a 3-mer to a 15-mer.

8. The method of any of claims 1 to 5, wherein the method yields an unnatural chondroitin sulfate oligosaccharide.

9. The method of any of claims 1 to 5, wherein the method yields a structurally defined chondroitin sulfate oligosaccharide.

10. The method of any of claims 1 to 5, wherein the method yields at least about 30 milligrams or more of chondroitin sulfate oligosaccharide.

11. The method of any of claims 1 to 5, wherein the synthetic chondroitin sulfate oligosaccharides are selected from chondroitin sulfate A (CS-A), chondroitin sulfate C (CS-C), chondroitin sulfate D (CS-D) and chondroitin sulfate E (CS-E).

12. The method of any of claims 1 to 11, wherein the synthetic chondroitin sulfate oligosaccharides comprise a structure selected from the group consisting of: 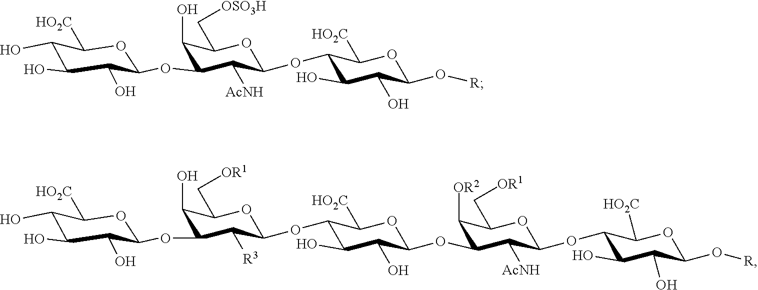
where R¹=SO₃H; R²=H; R³=NHAC,
where R¹=H; R²=H; R³=NHAC,
where R¹=R²=H; R³=NHFTA,
where R¹=SO₃H; R²=H; R³=NHFTA,
where R¹=R²=H; R³=NH₂,
where R¹=SO₃H; R²=H; R³=NH₂,
where R¹=R²=H; R³=N₃, or
where R¹=SO₃H; R²=H; R³=N₃; 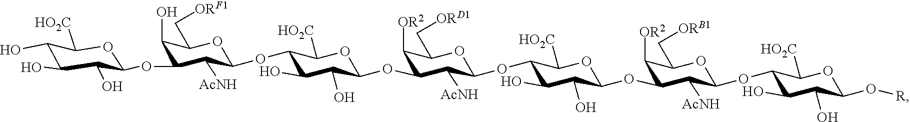
where R^{B1}=R^{D1}=R^{F1}=SO₃H; R²=H,
where R^{B1}=R^{D1}=R^{F1}=H; R²=SO₃H,
where R^{B1}=SO₃H; R^{D1}=R^{F1}=R²=H, or
where R^{B1}=R^{D1}=SO₃H; R^{F1}=R²=H; and 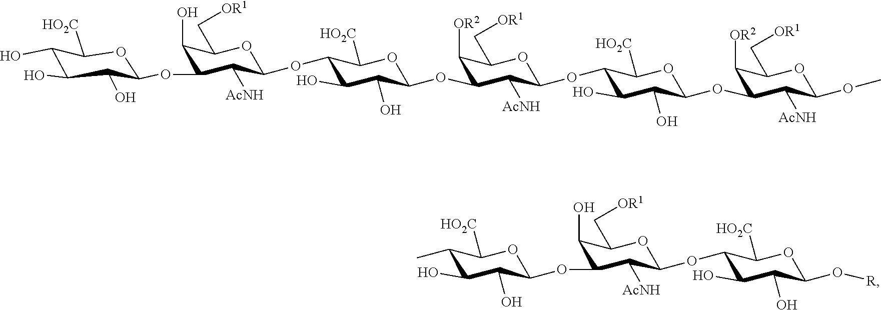
where R¹=SO₃H; R²=H, or
where R¹=H; R²=SO₃H,
wherein R is selected from the group consisting of —H, alkyl (such as but not limited to —CH₃ or —CH₂CH₃), substituted alkyl, aryl, and substituted aryl (such as but not limited to a p-nitrophenyl group).

13. The method of any of claims 1 to 11, wherein the synthetic chondroitin sulfate oligosaccharides comprise a structure selected from the group consisting of: 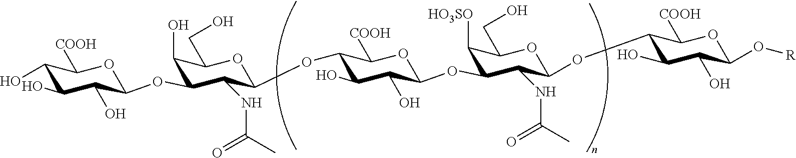
wherein n=4 (CS-A 11-mer), n=5 (CS-A 13-mer), n=6 (CS-A 15-mer), n=7 (CS-A 17-mer), or n=8 (CS-A 19-mer); 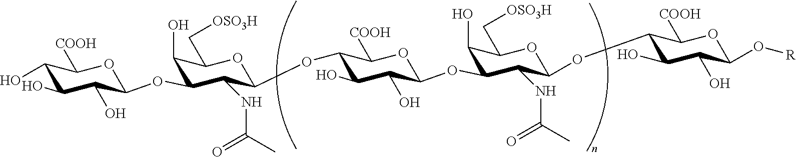
wherein n=4 (CS-C 11-mer), n=5 (CS-C 13-mer), n=6 (CS-C 15-mer), n=7 (CS-C 17-mer), or n=8 (CS-C 19-mer); and 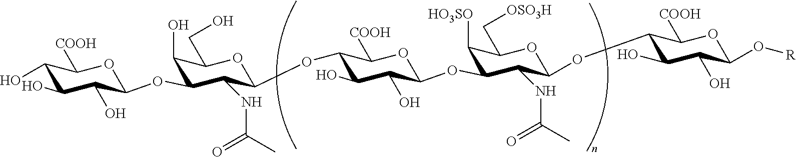
wherein n=1 (CS-E 5-mer), n=2 (CS-E 7-mer), n=3 (CS-E 9-mer), n=4 (CS-E 11-mer), n=5 (CS-E 13-mer), n=6 (CS-E 15-mer), n=7 (CS-E 17-mer) or n=8 (CS-E 19-mer),
wherein R is selected from the group consisting of —H, alkyl (such as but not limited to —CH₃ or —CH₂CH₃), substituted alkyl, aryl, and substituted aryl (such as but not limited to a p-nitrophenyl group).

14. A synthetic chondroitin sulfate oligosaccharide produced by a method of any of claims 1 to 13.

15. A library or panel of synthetic chondroitin sulfate oligosaccharides produced by a method of any of claims 1 to 13.

16. A synthetic chondroitin sulfate oligosaccharide, comprising a 4-O-sulfated and/or 6-O-sulfated chondroitin oligosaccharide having a size ranging from a 3-mer to a 15-mer.

17. The synthetic chondroitin sulfate oligosaccharide of claim 16, wherein the synthetic chondroitin sulfate oligosaccharides are selected from chondroitin sulfate A (CS-A), chondroitin sulfate C (CS-C), chondroitin sulfate D (CS-D) and chondroitin sulfate E (CS-E).

18. The synthetic chondroitin sulfate oligosaccharide of any of claims 16 to 17, wherein the synthetic chondroitin sulfate oligosaccharide is an unnatural chondroitin sulfate oligosaccharide.

19. A synthetic chondroitin sulfate oligosaccharide, wherein the synthetic chondroitin sulfate oligosaccharides comprise a structure selected from the group consisting of: 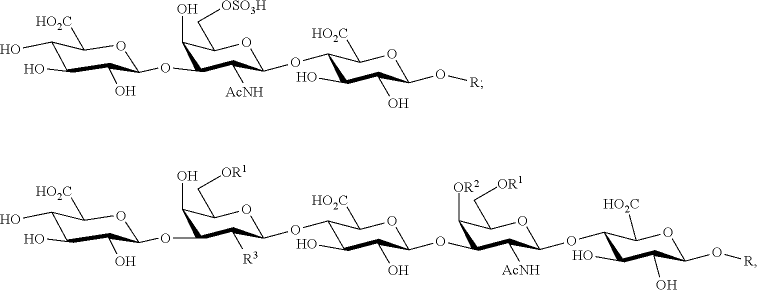
where R¹=SO₃H; R²=H; R³=NHAC,
where R¹=H; R²=H; R³=NHAC,
where R¹=R²=H; R³=NHFTA,
where R¹=SO₃H; R²=H; R³=NHFTA,
where R¹=R²=H; R³=NH₂,
where R¹=SO₃H; R²=H; R³=NH₂,
where R¹=R²=H; R³=N₃, or
where R¹=SO₃H; R²=H; R³=N₃; 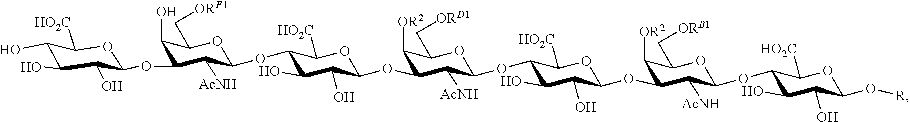
where R^{B1}=R^{D1}=R^{F1}=SO₃H; R²=H,
where R^{B1}=R^{D1}=R^{F1}=H; R²=SO₃H,
where R^{B1}=SO₃H; R^{D1}=R^{F1}=R²=H, or
where R^{B1}=R^{D1}=SO₃H; R^{F1}=R²=H; and 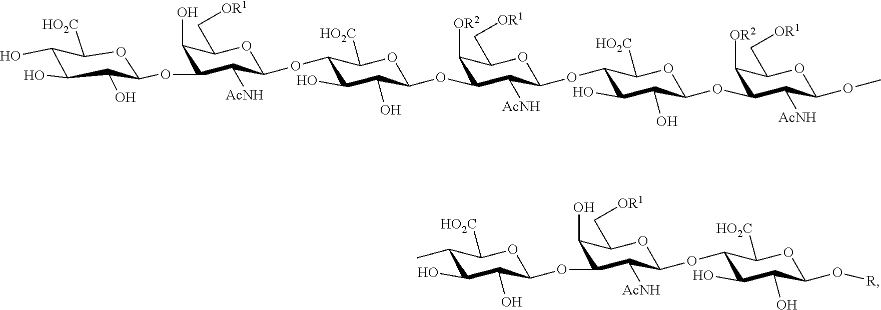
where R¹=SO₃H; R²=H, or
where R¹=H; R²=SO₃H,
wherein R is selected from the group consisting of —H, alkyl (such as but not limited to —CH₃ or —CH₂CH₃), substituted alkyl, aryl, and substituted aryl (such as but not limited to a p-nitrophenyl group).

20. A synthetic chondroitin sulfate oligosaccharide, wherein the synthetic chondroitin sulfate oligosaccharides comprise a structure selected from the group consisting of: 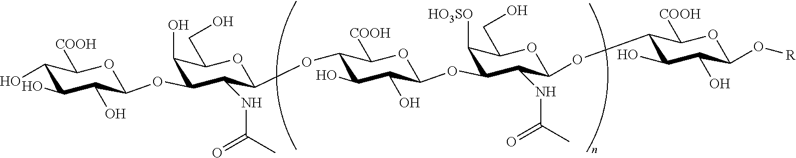
wherein n=4 (CS-A 11-mer), n=5 (CS-A 13-mer), n=6 (CS-A 15-mer), n=7 (CS-A 17-mer), or n=8 (CS-A 19-mer); 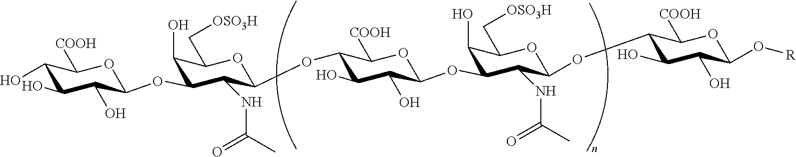
wherein n=4 (CS-C 11-mer), n=5 (CS-C 13-mer), n=6 (CS-C 15-mer), n=7 (CS-C 17-mer), or n=8 (CS-C 19-mer); and 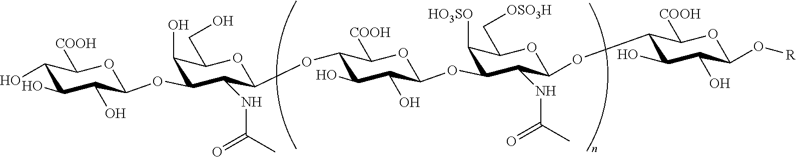
wherein n=1 (CS-E 5-mer), n=2 (CS-E 7-mer), n=3 (CS-E 9-mer), n=4 (CS-E 11-mer), n=5 (CS-E 13-mer), n=6 (CS-E 15-mer), n=7 (CS-E 17-mer) or n=8 (CS-E 19-mer),
wherein R is selected from the group consisting of —H, alkyl (such as but not limited to —CH₃ or —CH₂CH₃), substituted alkyl, aryl, and substituted aryl (such as but not limited to a p-nitrophenyl group).

21. A composition comprising one or more synthetic chondroitin sulfate oligosaccharides of any of claims 16 to 20.

22. The composition of claim 21, further comprising a pharmaceutically acceptable carrier.
